(19) United States
(12) Patent Application Publication
(10) Pub. No.: US 2009/0312725 A1
(43) Pub. Date: Dec. 17, 2009

(54) NEGATIVE PRESSURE WOUND THERAPY FLUID COLLECTION CANISTER

(76) Inventor: Richard M. Braga, Taunton, MA (US)

Correspondence Address: Tyco Healthcare Group LP;d/b/a Covidien, 15 Hampshire Street, Mansfield, MA, 02048, US

(21) Appl. No.: 12/139,041

(22) Filed: Jun. 13, 2008

(51) Int. Cl. A61M 1/00

(52) U.S. Cl. 604/313

(57) ABSTRACT

A system for subatmospheric pressure therapy in connection with healing a surgical wound includes a wound dressing dimensioned for positioning relative to a wound bed of a subject, a subatmospheric pressure mechanism having a vacuum source for presenting subatmospheric pressure at the wound bed and a collection canister. The collection canister includes a canister housing defining an internal chamber for collecting exudates from the wound bed removed under subatmospheric pressure, an inlet to receive the exudates for depositing in the internal chamber, an outlet in fluid communication with the vacuum source and a valve mounted adjacent the inlet. The valve is adapted to open in the presence of a vacuum condition within the internal chamber to permit the exudates to flow therethrough and to close in the absence of a vacuum condition within the internal chamber. The valve reduces the amount of exudates leakage, and eliminates the need for the use of clamping or the like to clamp about the system tubing. The collection canister may include a valve housing at least partially disposed within the internal chamber of the canister housing. The valve housing defines a valve seat having an opening in fluid communication with the inlet. The valve is mounted to the valve seat adjacent the opening and movable relative to the valve seat between a closed position occluding the opening of the valve seat and an open position at least partially exposing the opening in the valve housing. The valve may be a zero closure valve such an umbrella valve.

### BACKGROUND

1. Technical Field

The present disclosure relates to treating an open wound with application of subatmospheric pressure, and, more specifically, relates to an improved collection canister for use in a subatmospheric pressure therapy system.

2. Background of Related Art

Wound closure involves the migration of epithelial and subcutaneous tissue adjacent the wound towards the center and away from the base of the wound until the wound closes. Unfortunately, closure is difficult with large wounds, chronic wounds or wounds that have become infected. In such wounds, a zone of stasis (i.e. an area in which localized swelling of tissue restricts the flow of blood to the tissues) forms near the surface of the wound. Without sufficient blood flow, the epithelial and subcutaneous tissues surrounding the wound not only receive diminished oxygen and nutrients, but, are also less able to successfully fight microbial infection and, thus, are less able to close the wound naturally. Such wounds have presented difficulties to medical personnel for many years.

Negative pressure therapy also known as suction or vacuum therapy has been used in treating and healing wounds. Application of negative pressure, e.g. reduced or subatmospheric pressure, to a localized reservoir over a wound has been found to assist in closing the wound by promoting blood flow to the area, stimulating the formation of granulation tissue, and encouraging the migration of healthy tissue over the wound. Negative pressure may also inhibit bacterial growth by drawing fluids from the wound such as exudates, which may tend to harbor bacteria. This technique has proven particularly effective for chronic or healing-resistant wounds, and is also used for other purposes such as post-operative wound care.

Generally, negative pressure therapy provides for a wound to be covered to facilitate suction at the wound area. A fluid conduit provides fluid communication between the interior of a wound covering and an external vacuum source. Atmospheric gas, wound exudates, or other fluids may be drawn from the reservoir through the fluid conduit to stimulate healing of the wound. Exudates drawn from the reservoir may be deposited in a collection canister.

### SUMMARY

Accordingly, the present disclosure is directed to a system for subatmospheric pressure therapy in connection with healing a surgical wound. The system includes a wound dressing dimensioned for positioning relative to a wound bed of a subject, a subatmospheric pressure mechanism having a vacuum source for presenting subatmospheric pressure at the wound bed and a collection canister. The collection canister includes a canister housing defining an internal chamber for collecting exudates from the wound bed removed under subatmospheric pressure, an inlet to receive the exudates for depositing in the internal chamber, an outlet in fluid communication with the vacuum source and a valve mounted adjacent the inlet. The valve is adapted to open in the presence of a vacuum condition within the internal chamber to permit the exudates to flow therethrough and to close in the absence of a vacuum condition within the internal chamber. The valve reduces the amount of exudates leakage, and eliminates the need for the use of clamping or the like to clamp about the system tubing. The collection canister may include a valve housing at least partially disposed within the internal chamber of the canister housing. The valve housing defines a valve seat having an opening in fluid communication with the inlet. The valve is mounted to the valve seat adjacent the opening and movable relative to the valve seat between a closed position occluding the opening of the valve seat and an open position at least partially exposing the opening in the valve housing. The valve may be a zero closure valve such an umbrella valve.

Other features of the system and use thereof are also presented.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a view of the portable wound therapy system of the present disclosure illustrating the wound dressing, collection canister and the subatmospheric pressure mechanism;

FIG. 2 is view of the negative pressure wound therapy system of FIG. 1 further illustrating the collection canister;

FIG. 3 is a cross-sectional side view of the collection canister of FIG. 2;

FIG. 4 is a perspective view of an alternative embodiment of the collection canister of FIG. 2;

FIG. 5 is a perspective view of another alternative embodiment of the collection canister of FIG. 2; and

FIG. 6 is a top plan view of the valve of the collection canister of FIG. 2.

### DETAILED DESCRIPTION

The wound therapy system of the present disclosure promotes healing of a wound via the use of a wound dressing and a subatmospheric pressure mechanism. Generally, the subatmospheric pressure mechanism applies subatmospheric pressure to the wound to effectively remove wound fluids or exudates captured by the composite wound dressing and deposited in a collection canister, and to increase blood flow to the wound bed and enhance cellular stimulation of epithelial and subcutaneous tissue. The wound therapy system may be portable, i.e., it may be worn or carried by the subject such that the subject may be completely ambulatory during the therapy period, or the system may be non-ambulatory. The wound therapy system including the subatmospheric pressure mechanism and components thereof may be entirely disposable after a predetermined period of use or may be individually disposable whereby some of the components are reused for a subsequent therapy application.

The wound therapy system of the present disclosure promotes healing of a wound in conjunction with subatmospheric negative pressure therapy. The system may incorporate a variety of wound dressings, subatmospheric mechanisms including pressure sources and pumps, and collection canisters.

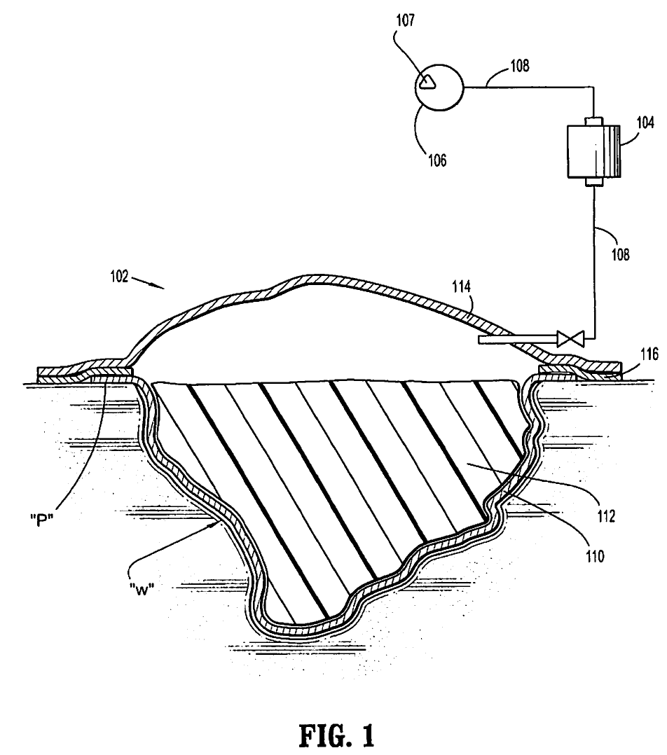

Referring now to FIG. 1, the wound therapy system 100 of the present disclosure is illustrated. Wound therapy system 100 includes composite wound dressing 102, collection canister 104 and subatmospheric pressure mechanism 106. In general, subatmospheric pressure mechanism 106 draws a vacuum through collection canister 104 and within wound dressing 102 through a conduit, identified schematically as reference numeral 108.

Wound dressing 102 may includes several components, namely, wound contact layer or member 110, a wound packing member or filler 112 supported by the contact member 110 and outer layer or cover member 114. Wound contact member 110 is adapted to substantially conform to the topography of a wound bed “w”. Wound contact member 110 may be substantially porous to permit exudates to pass from the wound bed “w” through the wound contact member 110.

Wound packing member 112 of wound dressing 102 is intended to absorb and/or transfer wound fluid and exudates. Exemplary absorbent materials include foams, nonwoven composite fabrics, hydrogels, cellulosic fabrics, super absorbent polymers, and combinations thereof. Typically, wound packing member 112 can contain or absorb up to about 100 cubic centimeters (cc) or more of wound fluid. Preferably, the absorbent material includes the antimicrobial dressing sold under the trademark KERLIX® AMD by Tyco Healthcare Group LP (d/b/a Covidien). In one preferred embodiment, packing member 112 could be preformed or shaped to conform to varying shapes of the wound bed. Those skilled in the art will recognize that packing member 112 can be formed in any suitable shape. Packing member 112 may include multiple layers. In another performed embodiment, the packing member 112 may be constructed in layers of varying absorbent materials to assist in directional flow or exudates away from the wound.

In the alternative, absorbent or non-absorbent packing member 112 may include a bead arrangement as disclosed in commonly assigned U.S. Patent Publication No. 2007/0185463, the entire contents of which is incorporated herein by reference. The beads disclosed in the '463 publication are preferably substantially rigid so as to maintain their shapes for at least a predetermined period of time during healing. The beads when arranged within the wound bed “w” define spaces or passages therebetween to permit wound exudates to pass through the passages. The sizes of the beads may vary, but they should be sized to achieve the proper pore size through the bead arrangement to facilitate cell proliferation and allow fluid and air to be evacuated from the wound. A porosity in the range of 10-1000 μm has been found beneficial in stimulating cell proliferation and in allowing fluid and air to be evacuated from the wound. The beads may work in conjunction with contact member 110 to conform to the wound bed “w” while allowing drainage of wound exudates and release of air from the wound bed “w” without clogging. As the negative pressure is applied, the beads will move and readjust their respective positions to prevent painful ingrowth that can occur with current foam dressing designs.

Referring still to FIG. 1, outer member or wound covering 114 encompasses the perimeter of the wound dressing 100 to surround wound bed “w” and to provide a liquid-tight seal around the perimeter “p” of the wound bed “w”. For instance, the sealing mechanism may be any adhesive bonded to a layer that surrounds the wound bed “w”. The adhesive must provide acceptable adhesion to the tissue surrounding the wound bed “w”, e.g., the periwound area, and be acceptable for use on the skin without contact deteriorization (e.g., the adhesive should preferably be non-irritating and non-sensitizing). The adhesive may be permeable or semi permeable to permit the contacted skin to breathe and transmit moisture. Additionally, the adhesive could be activated or de-activated by an external stimulus such as heat or a given fluid solution or chemical reaction. Adhesives include, for example, medical grade acrylics like the adhesive used with POLYSKIN™ II Transparent Dressing of Tyco Healthcare Group LP (d/b/a Covidien) or any silicone or rubber based medical adhesives that are skin friendly and non irritating. Wound covering member 114 may be provided with an adhesive backing and/or alternatively, an adhesive may be applied to the wound covering 112 and/or skin during the procedure.

Outer suitable wound dressing are disclosed in commonly assigned U.S. Patent Publication Nos. 2007/0078366, 2007/0066946 and 2007/0055209, the entire contents of each disclosure being incorporated herein by reference.

Subatmospheric pressure mechanism 106 of the system 100 may be portable, semi-portable or a component of a non-ambulatory system such as, a wall suction system in a hospital setting. Subatmospheric pressure mechanism 106 includes a vacuum source or pump 107 adapted to present a vacuum condition within collection canister 104 and wound dressing 102. The vacuum level to be achieved may be a range between about 35 mmHg and about 125 mmHg, or between about 50 mmHG and 80 mmHg. The vacuum source or pump may be a pump of the diaphragmatic, peristaltic or bellows type or the like, in which the moving part(s) draw exudates out of the wound bed “w” into the wound dressing 102 by creating areas or zones of decreased pressure e.g., vacuum zones with the wound dressing 100. This area of decreased pressure preferably communicates with the wound bed “w” to facilitate removal of the fluids therefrom and into the absorbent or non-absorbent packing member 110. The vacuum source or pump is actuated by an actuator which may be any means known by those skilled in the art, including, for example, AC motors, DC motors, voice coil actuators, solenoids, etc. Subatmospheric pressure mechanism 106 may include a power source such as alkaline batteries, wet cell batteries, dry cell batteries, nickel cadmium batteries, solar generated means, lithium batteries, NiMH batteries (nickel metal hydride) each of which may be of the disposable or rechargeable variety. Additional components of subatmospheric pressure mechanism may include a pressure sensor to monitor pressure adjacent the vacuum source or pump or selected locations displaced from the pump, and regulation or control means. The control means may incorporate a motor controller/driver including processing and drive circuitry to control or vary the drive voltage to the motor of the vacuum source or pump responsive to the pressure sensed by the pressure sensor. The output of the motor of the vacuum source may be increased or decreased, or initiated or discontinued, as controlled by control means. The pressure sensor would also provide information to assist in detecting a leak in the wound therapy system if the optimal subatmospheric pressure is not achieved. Regulation or control means may also have an alarm such as a visual, audio or tactile sensory alarm (e.g., vibratory etc.) to indicate to the user when specific conditions have been met (e.g., the desired vacuum level or loss of vacuum) as may be associated with a full exudates container. The pressure sensor may be replaced or supplemented with any other type of sensor or detector for measuring or detecting a characteristic or condition of the wound bed “w”. Additional sensors contemplated include thermal sensors, bacterial sensors, oxygen sensors, moisture sensors, etc. which will provide the clinician with additional diagnostic information.

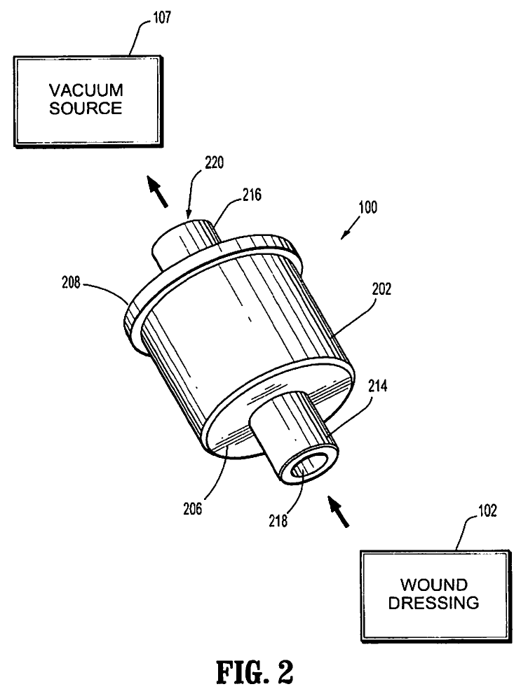

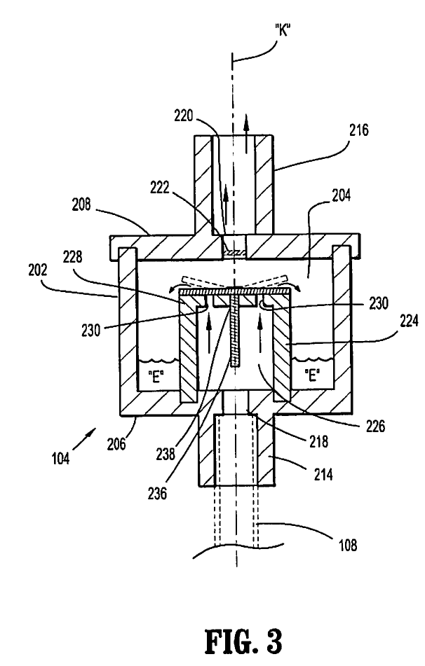

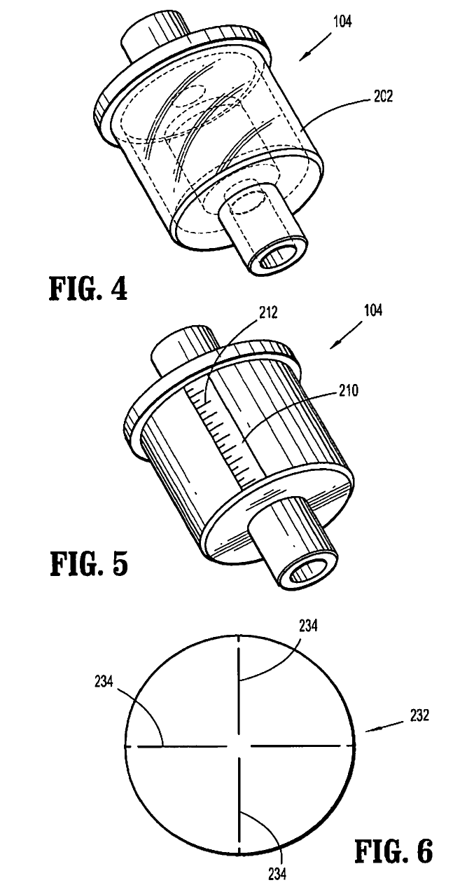

With reference to FIGS. 2 and 3, in conjunction with FIG. 1, further details of system 100 and collection canister 104 will be discussed. Collection canister 104 of system 100 includes canister housing 202 which defines internal chamber 204 for collection of the wound exudates. Canister housing 202 further defines longitudinal axis “k” and has opposed longitudinal end faces 206, 208. Canister housing 202 may be formed of plastic, polymer or other suitable material, and may be transparent, translucent or otherwise configured to permit viewing of the contents therein. FIG. 4 illustrates a transparent canister housing 202. Alternatively, as depicted in FIG. 5, canister housing 202 may include a viewing window 210 to enable viewing of the contents within internal chamber 204 of canister housing 202. Viewing window 210 may include gradations 212 or other markings for monitoring the level of exudates within canister housing 104.

Referring again to FIGS. 2 and 3, longitudinal end face 206 may be integrally formed with canister housing 202 and longitudinal end face 208 may be a separate component releasably connected to housing 102 via conventional means such as a threaded engagement or snap fit. The releasable connection of longitudinal end face 208 will permit the removal of longitudinal end face to define a releasable cover 208 thereby permitting emptying of collection canister 104. In the alternative, longitudinal end face or cover 208 may be integral with canister housing 202 for disposal of collection canister 104 after a single use. Longitudinal end faces 206, 208 may define respective extensions ports 214, 216 depending outwardly from canister housing 202. Extension port 214 defines an inlet 218 to receive exudates removed from wound dressing 102. Extension port 214 may connect to tubing 108 extending from wound dressing 102 to establish fluid communication between the wound dressing 102 and canister housing 104. Extension port 216 defines a vacuum or outlet 220 port adapted to establish fluid communication with vacuum pump 107 of subatmospheric pressure mechanism via tubing 108 (FIG. 2).

Vacuum source 107 and/or tubing 108 may be integrally formed with, securely affixed to, and/or selectively attached to respective extension ports 214, 216. A hydrophobic membrane 222 may cover outlet port 220 to prevent exudates “E” from being drawn into vacuum source 107.

Collection canister 104 further includes valve housing 224 which is disposed within internal chamber 204. Valve housing 224 may be coaxially arranged within canister housing 104 with respect to longitudinal axis “k”. Valve housing 106 may be monolithically formed with canister housing 202 or may be a separate component secured within, and to, canister housing 202 through conventional means. Valve housing 224 may be a substantially cylindrical body and defines internal valve chamber 226. Valve chamber 226 is in fluid communication with inlet port 218 of collection canister 218 of housing 202. Valve housing 224 further defines valve seat 228 in longitudinal opposed relation relative to inlet port 218. Valve seat 228 includes a plurality of openings 230 which communicate with valve chamber 226 of valve housing 224 and internal chamber 204 of canister housing 202. Valve seat 228 may include any number of openings 230 configured in any arrangement.

Valve housing 224 further includes valve 232 which is arranged in juxtaposed relation with valve seat 228. Valve 232 may be composed of rubber, plastic, polymer or any other suitable material. Valve 232 is adapted to move between an initial or rest position, e.g., in the absence of a vacuum condition within internal chamber 204 of canister housing 202 closing off openings 230 of valve seat 228 to prevent flow of exudates or fluids including air in either direction, and an open or actuated position shown in phantom in FIG. 3, e.g., in the presence of a vacuum condition within the internal chamber 204 of the canister housing 202, permitting flow of exudates and other fluids through openings 230. Valve 232 may be a septum valve, umbrella valve, duck bill valve or any other zero closure valve which can move between substantially open and closed positions. In one embodiment, depicted in the top plan view of FIG. 6, valve 232 is an umbrella type valve and may define at least one radial slit to assist in transitioning between the closed and open positions. A plurality of radial slits 234 is also envisioned. Valve 232 may be secured to valve seat 228 through any conventional means including via screws, fasteners or the like. In one embodiment, valve 234 includes valve stem 236 which extends through central opening 238 of valve seat 228. Valve stem may be secured within opening 238 through a snap fit, adhesives, cements or the like.

In operation, collection canister 104 is coupled to wound dressing 102 via tubing 108 and to vacuum source 107 of subatmospheric pressure mechanism 106 via tubing 108 to establish fluid communication. Prior to activation of vacuum pump 107 of subatmospheric pressure mechanism 106, e.g., in the absence of a vacuum condition within internal chamber 204, valve 232 is engaged with valve seat 228 effectively covering openings 120. Subatmospheric pressure mechanism 104 is then activated whereby vacuum source or pump 107 draws a vacuum within internal chamber 204 of canister housing 202. In the presence of the vacuum condition within internal chamber 204, valve 232 flexes or deforms (shown in phantom, FIG. 3) to move from its initial condition to its activated position displaced from valve seat 228. In this condition, the vacuum is communicated through openings 230, valve chamber 226 and wound dressing 102 to draw exudates within internal chamber 204 of canister housing 202. Exudates are collected in internal chamber 204 of collection canister 104. Exudates “E” continue to flow into internal chamber as long as a vacuum is provided to housing 102. In one mode of therapy, vacuum source or pump 107 is operated continuously at a predetermined pressure level, e.g., at between about 35 mmHg and 125 mmHg. In another continuous mode of treatment, vacuum pump 107 is programmed through logic to cycle between a preset maximum value (of between about 50-80 mmHg) and a minimum value of e.g., about 20-30 mmHg. Other modalities of therapy are also envisioned.

When subatmospheric pressure mechanism 106 and vacuum source 107 are deactivated, e.g., either manually, during a preprogrammed mode of therapy or due to system malfunction, valve 232 is no longer subject to a vacuum condition. In this condition, valve 232 assumes its initial state resting against valve seat 228 with openings 230 being occluded by the valve 232, thereby preventing exudates from leaking from internal chamber 204 of canister housing 202 back toward wound dressing 102. Accordingly, valve 232 self closes to occlude openings 230 of valve seat 228. Thus, no clamps or additional mechanisms are required to clamp the tubing 108 to prevent retrograde flow of the exudates. In addition, it is envisioned that the vacuum forces created within valve chamber 226 and within tubing 108 may facilitate drawing of valve 232 towards valve seat 228 to assist in sealing openings 230. The vacuum created by exudates “E” and the surface tension of exudates “E” within tubing 108 may prevent exudates “E” from leaking out of tubing 108.

Although the illustrative embodiments of the present disclosure have been described herein with reference to the accompanying drawings, it is to be understood that the disclosure is not limited to those precise embodiments, and that various other changes and modifications may be effected therein by one skilled in the art without departing from the scope or spirit of the disclosure.

What is claimed is:

1. A system for subatmospheric pressure therapy in connection with healing a surgical wound, which comprises:
a wound dressing dimensioned for positioning relative to a wound bed of a subject;
a subatmospheric pressure mechanism including a vacuum source for presenting subatmospheric pressure at the wound bed; and
a collection canister including:
a canister housing defining an internal chamber for collecting exudates from the wound bed removed under subatmospheric pressure;
an inlet to receive the exudates for depositing in the internal chamber;
an outlet in fluid communication with the vacuum source; and
a valve mounted adjacent the inlet, the valve adapted to open in the presence of a vacuum condition within the internal chamber to permit the exudates to flow therethrough and to close in the absence of a vacuum condition within the internal chamber.

2. The system for subatmospheric pressure therapy according to claim 1 wherein the collection canister includes a valve housing at least partially disposed within the internal chamber of the canister housing, the valve housing defining a valve seat having an opening in fluid communication with the inlet, the valve mounted to the valve seat adjacent the opening and movable relative to the valve seat between a closed position occluding the opening of the valve seat and an open position at least partially exposing the opening in the valve housing.

3. The system for subatmospheric pressure therapy according to claim 1 wherein valve is an umbrella valve.

4. The system for subatmospheric pressure therapy according to claim 1 including a hydrophobic membrane covering the outlet.

5. The system for subatmospheric pressure therapy according to claim 1 wherein the housing includes a transparent section.

6. The system for subatmospheric pressure therapy according to claim 1 wherein the housing includes a window for viewing the contents therein.

7. The system for subatmospheric pressure therapy according to claim 5 wherein the housing includes gradations for measuring the amount of exudates therein.
